(19) United States
(12) Patent Application Publication
(10) Pub. No.: US 2024/0290488 A1
(43) Pub. Date: Aug. 29, 2024

(54) SYSTEMS AND METHODS FOR DETERMINING ORAL MICROBIOME COMPOSITIONS AND HEALTH OUTCOMES

(71) Applicant: BRISTLE, INC., San Francisco, CA (US)

(72) Inventors: David LIN, Dublin, CA (US); Daniel GRANNICK, San Francisco, CA (US)

(73) Assignee: BRISTLE, INC., San Francisco, CA (US)

(21) Appl. No.: 18/571,673

(22) PCT Filed: Jun. 24, 2022
(86) PCT No.: PCT/IB2022/055899

§ 371 Date: Dec. 18, 2023

Related U.S. Application Data
(60) Provisional application No. 63/215,407, filed on Jun. 25, 2021.

(51) Int. Cl. G16H 50/20; C12Q 1/689; G16B 30/10

(52) U.S. Cl. CPC G16H 50/20; C12Q 1/689; G16B 30/10

(57) ABSTRACT

Methods and system for determining the oral microbiome composition of a subject and determining health outcomes are disclosed. In various embodiments, oral samples from subjects may be processed to generate oral microbial sequencing data, which is then analyzed with behavioral and/or clinical factors to predict health outcomes for the subject. Intervention plans based on the predicted health outcomes can also be generated for adaption by the subjects.

### CROSS-REFERENCE TO RELATED APPLICATIONS

This application is a National Phase under 35 U.S.C. 371 claiming the benefit of PCT/IB2022/055899, filed Jun. 24, 2022, which claims the benefit of U.S. Provisional Application No. 63/215,407, filed on Jun. 25, 2021, titled “Systems and Methods for Determining Oral Microbiome Compositions and Health Outcomes,” the disclosures of which are incorporated herein by reference in their entirety.

### TECHNICAL FIELD

The subject matter described herein relates to devices, methods, and systems for improving health and dental outcomes and, more particularly to devices, methods and systems for monitoring oral microbiome profiles and linking those profiles to possible health outcomes.

### BACKGROUND

Historically, diagnosis of oral disease occurs in dental offices using visual inspection and imaging techniques (e.g. x-rays). These techniques are useful in that they evaluate symptoms of underlying health conditions and then symptoms can be treated accordingly.

What is needed in the field are techniques to evaluate the cause of the symptoms or, in various cases, evaluate possible future health concerns before symptoms become apparent. Such techniques have the potential to predict, diagnose and/or treat future negative health outcomes before they reach a disease state. The systems and methods disclosed herein address this need by evaluating the components of oral microbiomes and using that information for downstream analysis to predict possible future health outcomes and inform oral care or treatment recommendations based on the predictions.

### BRIEF DESCRIPTION OF THE DRAWINGS

Illustrative embodiments of the present disclosure will be described with reference to the accompanying drawings, of which:

FIG. 1 is a flowchart depicting a method for identifying expected health outcomes and determining intervention plans for addressing the expected health outcomes according to various embodiments.

FIG. 2 is a flowchart depicting a method for nucleotide sequencing an oral sample according to various embodiments.

FIG. 3 is a flowchart depicting a method for determining a health outcome from analyzing sequencing data from an oral sample according to various embodiments.

FIG. 4 is a schematic diagram of a workflow for determining oral microbiome compositions and health outcomes according to various embodiments.

FIG. 5 is a bar graph displaying relative abundance values for species in an oral microbiome according various embodiments.

FIG. 6 is a sunburst chart displaying potential health outcomes for a subject according to various embodiments.

FIG. 7 is a tree diagram displaying potential health outcomes for a subject according to various embodiments.

FIG. 8 depicts a table comparing individual microbe distributions for a subject to a population according to various embodiments.

FIG. 9 is a scatter plot comparing a data from an individual to combined, average data over a population according to various embodiments.

FIG. 10 is a bar graph displaying potential health outcomes for a subject according to various embodiments.

FIG. 11 depicts a plot showing a microbial richness distribution according various embodiments.

FIG. 12 is a depiction of a pathogenic distribution of a subject compared to a population according to various embodiments.

FIG. 13 depicts several plots comparing individual microbe distributions for a subject to a population according to various embodiments.

FIG. 14 depicts a plot comparing a diversity score for a subject to a diversity score of a population according to various embodiments.

FIG. 15 is a depiction of a dysbiosis score of a subject compared to a population according to various embodiments.

FIG. 16 is a cartoon conveying information relating microbes to disease states according to various embodiments.

FIGS. 17A-17B are example plots showing predictions generated by the methods disclosed herein for periodontal disease (FIG. 17A) and dental caries (FIG. 17B) according to various embodiments.

FIG. 18 is a schematic diagram of a computer device/analytics server for processing sequencing data derived from an oral sample according to various embodiments.

FIG. 19 is a schematic diagram of a computer system for carrying out the methods provided herein according to various embodiments.

### DETAILED DESCRIPTION

Before the present embodiments are described in greater detail, it is to be understood that the embodiments are not limited to specific or particular embodiments described, as such may, of course, vary. It is also to be understood that the terminology used herein is for the purpose of describing particular embodiments only, and is not intended to be limiting, since the scope of the present invention will be limited only by the appended claims.

Embodiments for systems, methods of use, and apparatuses for determining oral microbiome compositions and health outcomes are described in the accompanying description and figures. In the figures, numerous specific details are set forth to provide a thorough understanding of certain embodiments. A skilled artisan will be able to appreciate that the systems, methods, and apparatuses described herein may be used in a variety of ways and circumstances, not limited, to what is specifically detailed. That is, while the present teachings are described in conjunction with various embodiments, it is not intended that the present teachings be limited to such embodiments. On the contrary, the present teachings encompass various alternatives, modifications, and equivalents, as will be appreciated by those skilled in the art. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences may be varied and remain within the spirit and scope of certain embodiments.

Where a range of values is provided, it is understood that each intervening value, to the tenth of the unit of the lower limit, unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

All publications mentioned herein are expressly incorporated herein by reference to disclose and describe the methods and/or materials in connection with which the publications are cited.

Logical operations making up the embodiments of the technology described herein are referred to variously as operations, steps, objects, elements, components, or modules. Furthermore, it should be understood that these may occur or be performed in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the technologies disclosed herein. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Although various embodiments of the claimed subject matter have been described above herein a certain degree of particularity, or with reference to one or more individual embodiments, those skilled in the art could make numerous alterations to the disclosed embodiments without departing from the spirit or scope of the claimed subject matter. Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

### I. Workflows for Determining Oral Microbiome Compositions and Health Outcomes

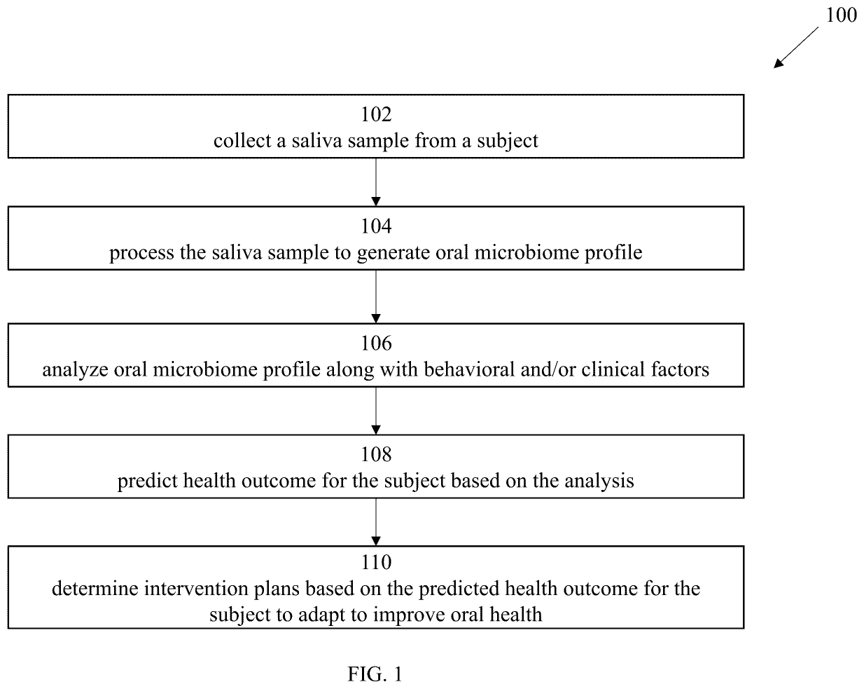

In various embodiments, FIG. 1 shows a flowchart 100 depicting a method for identifying expected health outcomes of a subject based on analysis of the subject's oral microbiome profile, clinical factors, behavioral factors, etc., and determining intervention plans for addressing the expected health outcomes based on the analysis. As illustrated, flowchart 100 includes a number of enumerated steps, but aspects of flowchart 100 may include additional steps before, after, and in between the enumerated steps. In various embodiments, one or more of the enumerated steps may be omitted or performed in a different order.

At 102, in some aspects, an oral sample from the subject's oral cavity may be collected using a oral sample collection kit. The collection kit may be configured to allow a subject to collect the oral sample in an unsupervised setting. In some instances, the collection kit may be configured to allow a subject to collect the oral sample in a supervised setting. For example, the collection kit may be configured to collect oral samples from multiple subjects (e.g., in a setting supervised by a professional qualified to collect the samples (e.g., health technician)).

Examples of an oral sample include saliva samples, plaque, etc. Saliva samples can be specific to a location (e.g., samples from tongue, crevicular/gingival fluid, etc.) or collection method (e.g., samples obtained from oral rinse, pure saliva, sponge, etc.). The oral samples can include microbiome including but not limited to bacteria, fungus, virus, archaea, parasites, etc. In some embodiments, the terms “oral sample” and “saliva sample” may be used interchangeably. Further, the terms “subject” and “patient” may be used interchangeably. In some embodiments, the term “microbiome” can refer to the identity and relative abundance of microbes in a given sample.

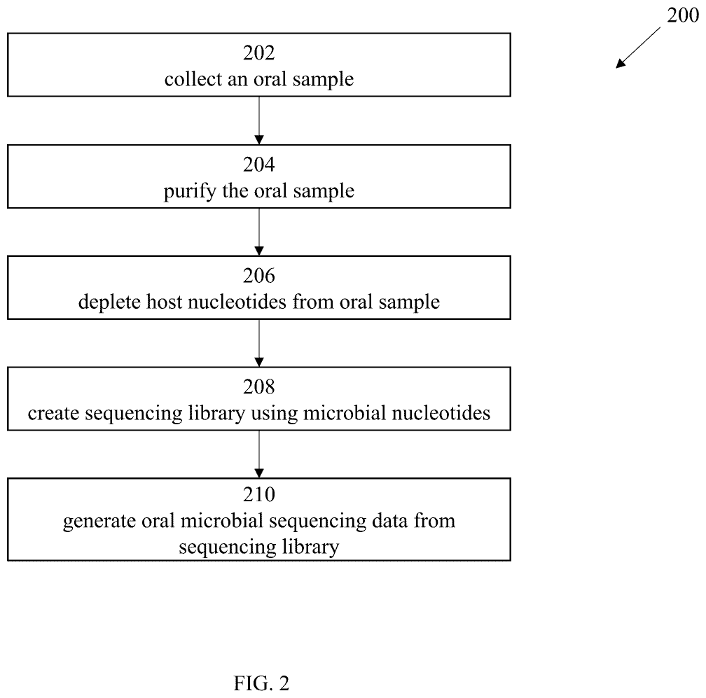

At 104, in various embodiments, the oral sample of the subject may be processed to extract the oral genomic sequencing data. In various embodiments, as shown in flowchart 200 of FIG. 2, the processing of the oral sample to generate the oral microbial sequencing data may include the purification, at 204, of the oral sample to concentrate host and microbial nucleotides after the oral/saliva sample is collected or obtained, at 202. For example the oral sample may be purified using a bead-based or a column-based total nucleotide purification technique. In some instances, the term “host” may be understood to refer to the “subject” (e.g., “host nucleotides” may refer to the nucleotides of the host/subject from which the oral sample is obtained).

Further, at 206, the host nucleotides, or portion thereof, may be depleted. For example, nucleic acid may be extracted/isolated from the oral sample through a chemical lysis technique, a physical lysis technique, or combination thereof. An example of a chemical lysis technique is detergent-based lysis technique and an example of a physical lysis technique is bead-bashing-based lysis technique. The depletion of the host nucleotides from the oral sample can utilize distinct differences in eukaryotic and prokaryotic biochemistry and nucleic acid composition to selectively enrich for microorganisms contained within the oral sample. In some instance, the depletion of the host nucleotides may include size selection, i.e., may be based on size of the nucleotides. In some instances, the depletion technique can be based on methylation of DNA. Eukaryotic DNA is methylated differently than DNA from prokaryotes, or phage, or archaea. This allows host DNA to be cut by methylation dependent restriction enzymes. This cutting makes the DNA shorter on average. In such cases, bead-based size selection can be used to remove the short fragments.

In various embodiments, the processing of the oral sample may also include the preparation or construction, at 208, of a sequencing library using the microbial nucleotides. In some instances, the construction of the sequencing library may include fragmenting the nucleic acids within the oral sample and attaching unique barcodes for subsequent de-multiplexing and identification of the oral sample and adapters for analysis via a sequencing platform. For instance, the sequencing library may be prepared using a technique that fragments and tags (“tagments”) input DNA or a ligation-based sequencing library preparation technique. At 210, in various embodiments, the oral microbial sequencing data may be generated from this sequencing library. The oral microbial sequencing data may be computationally demultiplexed, trimmed for quality control and subsequently analyzed to determine the identities of the organisms comprising the oral sample. As illustrated, flowchart 200 includes a number of enumerated steps, but aspects of flowchart 200 may include additional steps before, after, and in between the enumerated steps. In various embodiments, one or more of the enumerated steps may be omitted or performed in a different order.

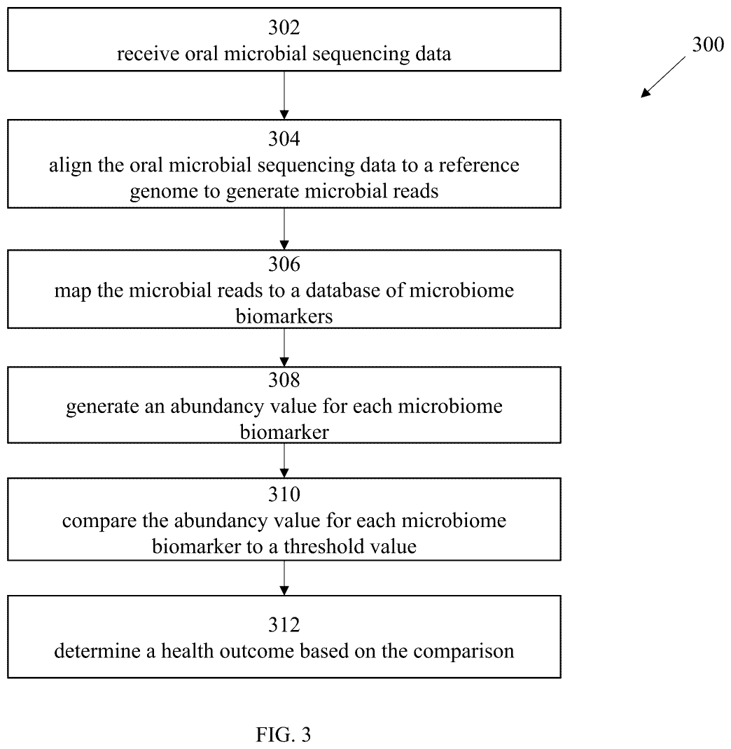

Returning to FIG. 1, in various embodiments, at 106, the oral microbiome profile may be analyzed, in some instances along with subject data related to behavioral and/or clinical factors of the subject from whom the oral sample is collected. Further, at 108, the health outcome of the subject may be predicted or determined based on the analysis. In various embodiments, FIG. 3 shows an example flowchart depicting a method for determining a health outcome from analyzing the oral microbiome sequencing data. In various embodiments, at 302, the oral microbial sequencing data may be received, and at 304, aligned to a reference genome to count or obtain microbial reads. The microbial reads may be aligned to human (e.g., hg38 reference genome) and microbial reference genome to differentiate between data associated with host and data associated with microbiome using a variety of packaged and custom software tools (e.g., bwa, bowtie, diamond, hs-blastn, blast, blastx, etc.).

At 306, in various embodiments, the microbial reads may be mapped to a database of microbiome biomarkers. In some instances, the microbiome biomarkers can be specific to a particular taxonomic group of the microbiome. For example, the biomarkers can be specific to a genus of the microbiome, a specie of the microbiome, a strain of the microbiome, a family of the microbiome, etc., or combination thereof. In such cases, the database to which the microbial reads are mapped may be a database of microbiome genus-specific biomarkers, a database of microbiome specie-specific biomarkers, a database of microbiome strain-specific biomarkers, a database of microbiome family-specific biomarkers, or a combination thereof.

In various embodiments, biomarkers can include any sequence from a given sample or specimen. For example, the microbial read itself or a k-mer from the microbial read can be biomarkers. In various embodiments, the biomarkers can be or include marker-genes (e.g., marker genes specific to a microbiome taxonomic group). For example, the database of microbiome biomarkers to which the microbial reads are mapped may include genus-specific, specie-specific, strain-specific, family-specific etc., marker-genes. The microbial reads may then be mapped to such databases to determine the count of aligned reads to specific microorganisms comprising the microbiomes, the taxonomic groups of microbiomes, etc., of the oral or saliva sample. That is, in sone instances, the count is the number of microbial reads from a given sample that align to a genome or part thereof in the reference database.

The mapping techniques may leverage marker-genes unique to specific organisms as defined in the database to determine an identification (ID) of the microbiome or a taxonomic group (e.g., genus, species, strain, family, etc.) of the microbiome, and relative abundance of the microorganisms. The relative abundance or composition of the microbiomes in the oral or saliva sample of the subject may be determined based on the mapping between the microbial reads and the database of microbiome biomarkers. The microbial reads can be mapped by software such as Metaphlan, CLARK, mOTUs, Metagenomic Intra-Species Diversity Analysis System (MIDAS), Kraken2, or other alignment tools that measure the counts of reads for shotgun metagenomics. The database may be generated for alignment using Metaphlan, MIDAS, Kraken2, or other custom software builds. In various embodiments, relative abundance is a measure that estimates the microbiome community based on the number of microbial reads that map to a particular organism. Depending on the method of alignment and the database, an algorithm may be applied to normalize the data across samples and microbes. Because microbes have different sized genomes, such process takes the different lengths into account. Additionally, if microbial reads align ambiguously, an estimation may be applied to correct for the ambiguity.

In various embodiments, at 308, an abundancy value may be computed or generated for each microbiome or microbiome biomarker based on the mapping of the microbial reads to the database of microbiome biomarkers. In some instances, the abundancy value may be computed or generated for each taxonomic group of a microbiome or each microbiome biomarker specific to a particular taxonomic group. For example, an abundancy value may be generated for each of the genus, specie, strain, family, etc., of a microbiome (e.g., bacteria, fungus, virus, etc.). The computed abundancy values may be provided as output files that include an oral microbiome profile of the subject. The oral microbiome profile may include the composition of the microbiomes in the oral sample as determined by the computed abundancy values of the microbiomes. For example, the oral microbiome profile may include the computed abundancy values and/or the relative abundance of a microbiome, which is the count of microbial reads that come from that microbiome normalized to the total microbial reads.

The output files from read alignment and mapping may then be visualized as tables, charts, graphs, or any other form of data visualization of microbiome profiles. For example, the microbiome profiles in the output files may include host and microorganism data, such as but not limited to relative abundance, functional profile and characteristics, species, strain, genus, family, etc., identification within an individual sample.

In various embodiments, at 310, the abundancy values for each microbiome may be compared to respective threshold value, and at 312, a health outcome for the subject of the oral sample may be determined based on these comparisons. That is, the abundancy value can be for a microbiome, a type (e.g., a taxonomy group) of the microbiome, etc., and health outcome for the subject of the oral sample can be determined by comparing the abundancy values to threshold values. In some aspects, threshold values may indicate the degree of positive or negative effect of a given microbe or set of microbes on health or disease based on whether the microbe or set of microbes are beneficial/non-pathogenic or pathogenic. For example, an abundancy value that is greater than a threshold value may indicate a positive health outcome when the microbiome type is beneficial for the oral health of the subject. For instance, the microbiome can be commensal bacteria associated with a minimal abundancy threshold below which the oral health of the subject is deemed to be unhealthy. In such cases, an abundancy value of commensal bacteria (e.g., or a taxonomic group thereof) that is no less than the minimal abundancy threshold may indicate a positive health outcome for the subject.

Alternatively, an abundancy value that is less than the abundancy threshold may indicate a negative health outcome when the microbiome type is beneficial for the oral health of the subject. In some instances, the opposite may be the case, i.e., an abundancy value that is greater than a threshold value may indicate a negative health outcome when the microbiome is pathogenic, i.e., harmful to the oral health of the subject, and the threshold value indicates microbiome abundancy above which the oral health of the subject is deemed to be unhealthy. Alternatively, an abundancy value that is no greater the threshold value may indicate a positive health outcome when the microbiome type is pathogenic. Examples of negative health outcomes include dental caries, halitosis, periodontitis, and/or the like.

In various embodiments, the threshold values associated with a microbiome may be pre-defined. For instance, the threshold values may be obtained from published/peer-reviewed literature and may represent consensus threshold values of the scientific community studying oral health issues. In some instances, the threshold values may be determined based on analysis of oral samples of a group of subjects as discussed in the instant specification. In various embodiments, biomarkers may be identified and associated with disease or other health-related outcomes by analyzing aggregate data from a group of subjects and comparing that to clinically diagnosed subject values. Relationships between biomarkers and oral diseases may be classified based on a comparison between the group of subjects and clinically diagnosed subject data sets to ensure statistically significant causal or correlative relationships. Once a biomarker is identified, the threshold value for that biomarker can be determined by assessing the relative abundance across subjects and clinically diagnosed subjects in conjunction with clinical factors and real-world evidence to derive a relevant biomarker status. A biomarker that exceeds a threshold may indicate that a user is at higher risk for or have specific oral disease or health-related outcomes.

Returning to FIG. 1, in various embodiments, at 106, the oral microbiome profile may be analyzed, in some instances along with subject data related to behavioral and clinical factors related to the subject. As noted above, the oral microbiome profile can be derived or obtained from the oral microbial sequencing data. In some instances, the oral microbiome profile may be augmented with analysis of subject data related to behavioral factors, clinical factors, etc., and at 108, the health outcome of the subject of the oral sample may be predicted or determined based on an analysis of the oral microbiome profile and the subject data. Examples of subject data related to clinical factors of the subject include medical/disease history of the subject, self-reported symptoms, etc., and examples of subject data related to behavioral factors of the subject include lifestyle information (e.g., hygiene, exercise levels, social determinants of health, smoking status, etc.) and/or the like. Subject data indicating poor oral or overall health may contribute to a prediction of poor health outcome, and vice versa.

At 110, in various embodiments, intervention plans may be determined based on the predicted health outcome to assist the subject in improving their oral health. For example, if the predicted health outcome is negative, an intervention plan including lifestyle changes and/or administration of therapeutics may be complied for use by the subject to forestall or prevent the predicted negative health outcome. For example, the intervention plan can include information on maintaining or improving proper lifestyle choices (e.g., diet, hygiene, etc.), clinical interventions (e.g., therapeutics), etc. If the predicted health outcome is positive, the intervention plan may accordingly include recommendations designed to reinforce or improve the positive health outcome.

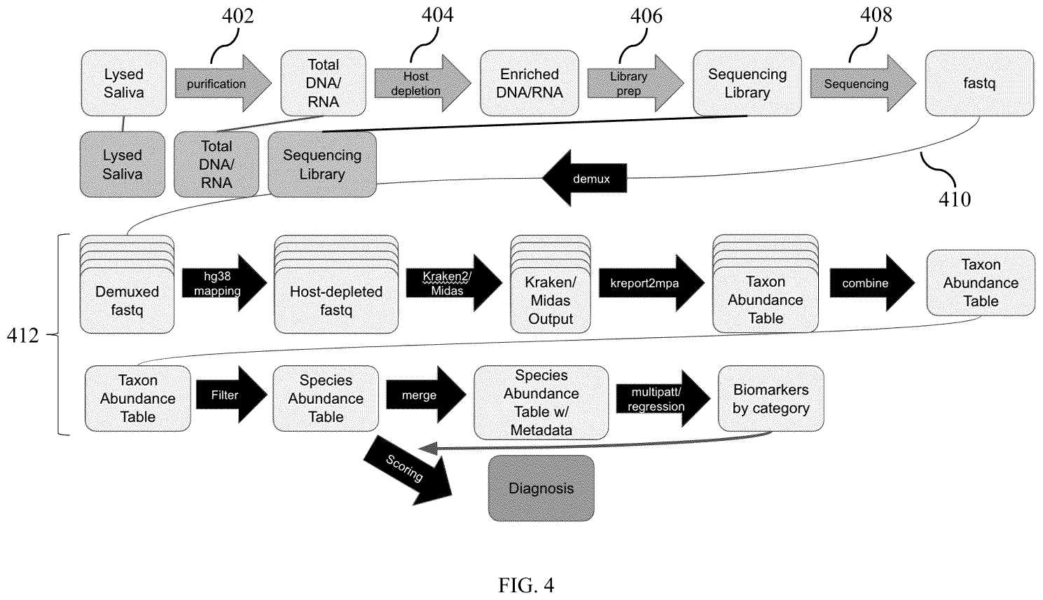

FIG. 4 is a schematic diagram of a workflow for determining oral microbiome compositions and health outcomes according to various embodiments. In various aspects, a method, which can be a k-mer-based classification method, for determining the oral microbiome composition of a subject is disclosed. In various embodiments, as discussed above, oral samples received from subjects may be processed for compatibility with genomic sequencing platforms to determine the nucleic acid sequence and composition of various microbiomes within the sample. Oral microbial sequencing data relating to the sample may then be generated from the a sequencing library created using microbial nucleotides of the sample. In various embodiments, the workflow for determining oral microbiome compositions and health outcomes comprises but is not limited to sample collection and receipt, nucleic acid extraction, sample processing and preparation, genomic sequencing, raw data collection, data processing, and data analysis. In various embodiments, raw microbial sequencing data for aggregate or individual samples may be received in FASTQ, FAST5, FASTQS, or any other suitable file formats for primary analysis, including demultiplexing libraries according to predetermined barcodes comprising unique identifier sequences affixed to samples during library preparation.

In various embodiments, a subject may provide an oral sample obtained from the oral cavity. For example, individual, multiple, or aggregate oral sample(s) from a subject or a set of subjects may be received in a collection kit that includes sample collection device suitable for obtaining and storing oral samples from oral cavities. Collection kits may be provided to subjects to allow collection of oral samples via unsupervised or supervised deposit of the oral samples into the sample collection devices of the collection kits.

In various embodiments, the oral or saliva sample collected from the subject may be preserved in buffers. For instance, buffers may be used during or after sample collection to allow or enhance preservation of the organisms in the oral sample and stabilize nucleic acids contained therein for improved downstream processing and analysis. The buffer may also act as a lysis mechanism to allow downstream isolation and processing of nucleic acids according to various embodiments. An example of a buffer is DNA/RNA shield reagent.

In various embodiments, at 402, the oral sample may be purified to concentrate host and microbial nucleotides. In some instances, as noted above, the oral sample may be lysed chemically and/or physically to allow extraction or isolation of the nucleic acid from the sample. The lysed oral sample may then be purified by a bead-based or a column-based total nucleotide purification technique, for example.

In various embodiments, at 404, the oral sample may be partially or completely depleted of host (e.g., human or subject) DNA through a series of sample processing steps to produce an oral sample that is enriched with microbial nucleotides (e.g., contains less or no host/human nucleotides). Host depletion leverages distinct differences in eukaryotic and prokaryotic biochemistry and nucleic acid composition to selectively enrich for microorganisms contained within the sample according to various embodiments.

In various embodiments, a process of host-depletion post-lysis that may rely on the biochemical differences between eukaryotic DNA and prokaryotic DNA may be employed. Eukaryotic DNA contains methylated cytosines at much higher frequency compared to prokaryotic DNA. Methods that can be used to reduce host DNA include Fc-MBD2 immunoprecipitation, anti-5-methylcytosine immunoprecipitation, or methyl-cytosine activated restriction digest followed by size selection, according to various embodiments. Size selection allows the isolation of nucleic acids associated with microbial species for downstream processing and sequencing.

In various embodiments, at 406, a sequencing library for generating oral microbial sequencing data of an oral sample may be prepared using microbial nucleotides of the sample. In some instances, oral samples are taken through a series of processing steps for compatibility with sequencing platforms used for nucleic acid analysis of the microbiome. In some aspects, library preparation comprises fragmentation of the nucleic acids within a sample and attachment of unique barcodes for subsequent demultiplexing and identification of samples and adapters for analysis via the sequencing platform according to various embodiments. In some instances, there may be multiple oral samples (e.g., from the same subject or a set of subjects), and the samples may be run individually or pooled prior to sequencing.

In various embodiments, the sequencing libraries may be prepared using Illumina Nextera® library preparation, or other library preparation methods. Library preparation may be miniaturized by modifications to the published Illumina Nextera® library prep methods through volume reduction or dilution of reagents. Further, multiple sequencing libraries may also be prepared in parallel. In some instances, each library may be sequenced individually or sequenced as a pool to reduce the sequencing of each library. In various embodiments, at 408, the oral microbial sequencing data may be generated based on the sequencing libraries. In some instances, the microbial sequencing data may be provided in FASTQ, FAST5, or any other suitable file format.

In various embodiments, at 410, the microbial sequencing data files containing the nucleic acid composition of pooled or individual oral samples may be computationally demultiplexed to identify the oral samples and characterize the associated microbiome profiles. In some instances, the unique barcodes affixed to samples may be utilized for said sample identification and characterization of the associated microbiome profiles. For example, the unique sequences of the barcodes can be used to identify and differentiate one or more samples from another. Further, the microbial sequencing data may then go through a quality control analysis to prepare the microbial sequencing data for downstream alignment, mapping, and microbiome profiling. In some instances, the quality control analysis may also include the trimming or removal of the adapter sequences and identifying barcodes affixed to the microbial sequencing data.

In various embodiments, processes 412 of FIG. 4 relate to the afore-mentioned alignment, mapping, and microbiome profiling of the microbial sequencing data. Although the mapping process of the processes 412 relates to the mapping of microbial reads to microbiome specie-specific biomarkers, it is to be understood that the processes 412 are non-limiting example embodiments and that the mapping of the microbial reads can be to any microbiomes or microbiome biomarkers specific to any taxonomic group (e.g., genus, species, strain, family, etc.) of microbiomes.

In various embodiments, at 412, the microbial sequencing data may be aligned to a reference genome to gather microbial reads from the microbial sequencing data. In some instances, the oral sample may not have been completely depleted of host (e.g., human or subject) nucleotides. In such cases, the microbial reads may be mapped to human and microbial reference genomes to differentiate between host and microbiome associated data. Further, the microbial reads may be mapped to a database of microbiome marker-genes, which in some cases can be specific to a taxonomic group (e.g., genus, species, strains, family, etc.) of the microbiome. In some instances, the mapping determines the count of aligned reads to specific organisms comprising the individual or aggregate sample. The methods here leverage marker-genes unique to specific organisms as defined in a database to determine the genus, strain, family, or species ID and relative abundance of microorganisms comprising the oral microbiome for a particular sample according to various embodiments. The microbial reads are mapped to the database of microbiome biomarkers by software such as Metagenomic Intra-Species Diversity Analysis System (MIDAS), Kraken2, or other alignment tools that measure the exact counts of reads that map to microbiome biomarkers (e.g., species specific marker-genes). The microbiome biomarkers (e.g., marker-genes) may be defined by database generation using MIDAS or Kraken2, or other custom-built softwares.

In various embodiments, the technologies for discovering oral biomarkers relevant to oral disease in a subject may comprise receiving oral genomic sequencing data, receiving an oral sample, collection methods and devices, buffer inclusion, host-DNA depletion methods, library preparation, demultiplexing, trimming, aligning the received sequencing data to a reference genome to gather microbial reads from the received sequencing data, mapping the gathered microbial reads to a database of species specific marker-genes, generating abundancy values of species selected from the group comprising bacterial species, fungal species, viral species, and combinations thereof, obtaining a relative value such as a percentage, putting the data into table format, report results and information to a subject, correlating linkages to disease or health outcomes, and comparing generated abundancy values of species to a threshold value for each species specific marker-gene.

In various embodiments, threshold values for individual or aggregate microbes based on species ID and relative abundance may be generated to associated microbiome signatures to expected clinical outcomes. Threshold values may indicate the degree of positive or negative effect for a given microbe or set of microbes on health or disease, or provide guidance on suitable interventions that may be used to aid or combat implicated microbes. In some aspects, the interventions can be behavioral and/or clinical interventions such as but not limited to interventions related to lifestyle (e.g., maintaining proper diet), hygiene (e.g., proper use of oral care products (e.g., toothpaste, floss, mouthwash, probiotics, etc.)), therapeutics (e.g., using active ingredients such as fluoride, nanohydroxyapatite, xylitol, etc.), and/or the like. Values are either derived from an analysis pipeline or are adapted from cited literature and validated.

### II. Visualization of Oral Microbiome Profiles

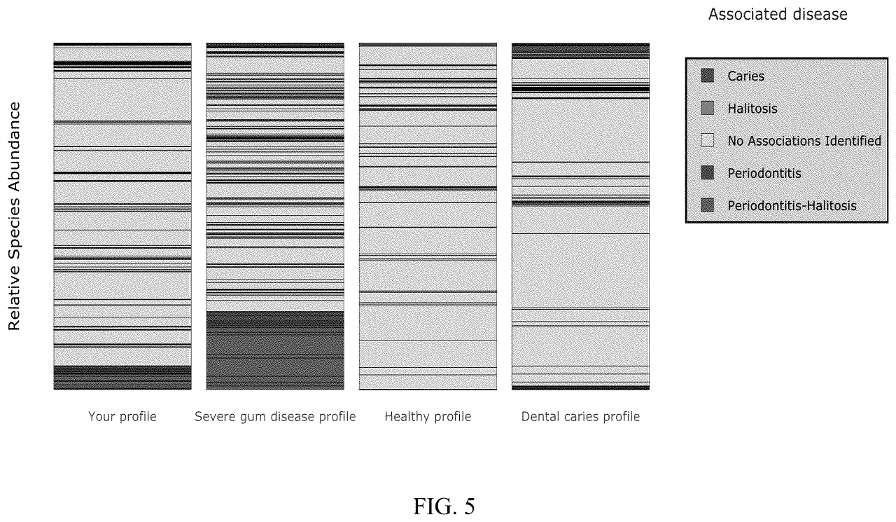

In various embodiments, as discussed above, systems and methods for generating an oral microbiome profile of an oral sample comprising abundancy values of microbiomes and/or taxonomic groups of microbiomes are disclosed. Examples of the microbiomes include but are not limited to bacteria, fungus, virus, etc., and the taxonomic groups include genus, species, strains, family, etc., of the microbiomes. In some instances, the oral microbiome profile may include the composition of the microbiomes as measured or quantified by the relative abundance of each microbiome with respect to the total amount of microbiomes (e.g., related to the total count of microbial reads). In some instances, the oral microbiome profile generated after read alignment and mapping may be presented in any form of data visualization, such as in a table, chart, graph, etc., as shown in FIGS. 5-17. The oral microbiome profile may include host and/or microorganism data, such as but not limited to the relative abundance, functional profile and characteristics, taxonomic group identification within an individual sample, and/or the like.

In various embodiments, the oral microbiome profile of a subject may be compiled in a subject report that presents the oral microbiome profile in the formats depicted in FIGS. 5-17. The oral microbiome profile is traditionally inaccessible as a tool for people to monitor their health. The systems and methods disclosed herein make the oral microbiome accessible to subjects for monitoring and maintaining their oral health (e.g., by reviewing the data contained in the subject report and adapting recommendations provided to address the subject's health outcome predicted based on the oral microbe profile). Through the workflow described herein, the salivary oral microbiome (or potentially swab or other sources) may be analyzed and the analyses may be presented to subjects in a way that is easily digestible. In some instances, the information related to the oral microbiome profile can be presented as interactive and/or dynamic plots through a web application, and the subject report can identify correlations between microbial abundance and disease status.

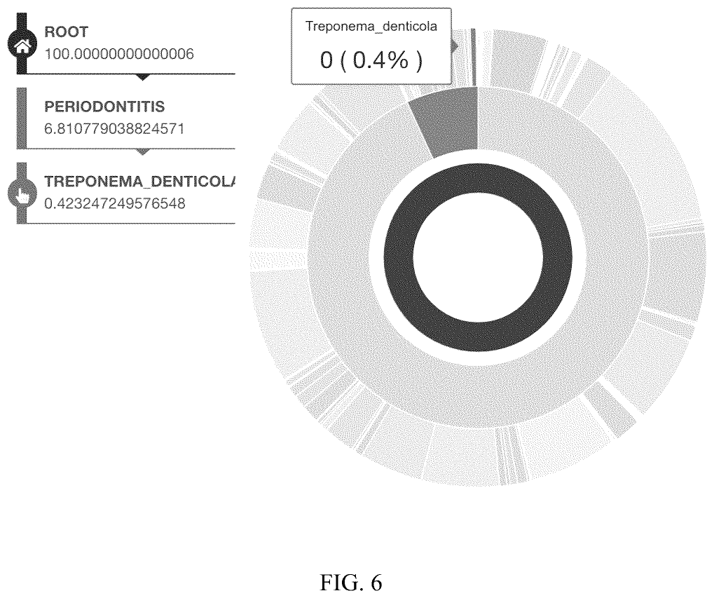

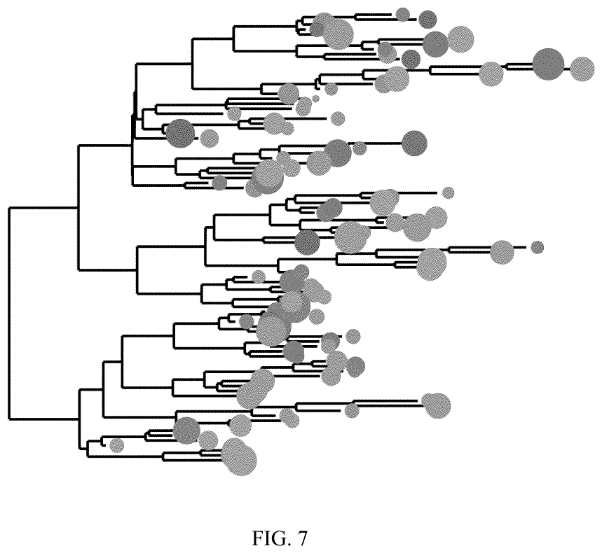

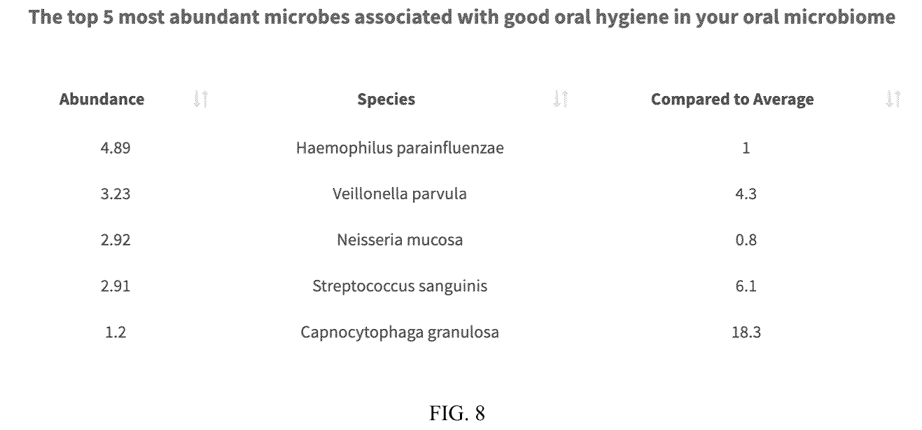

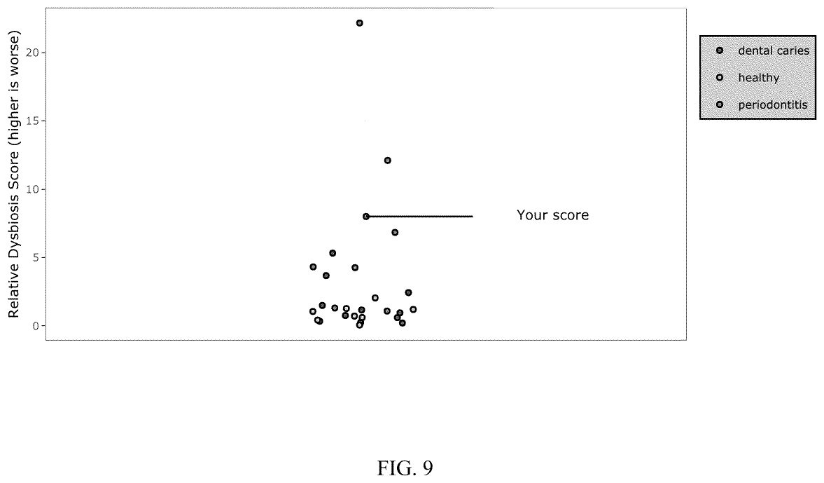

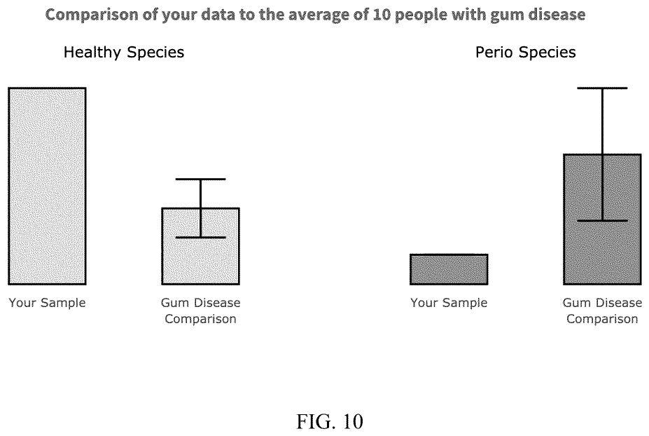

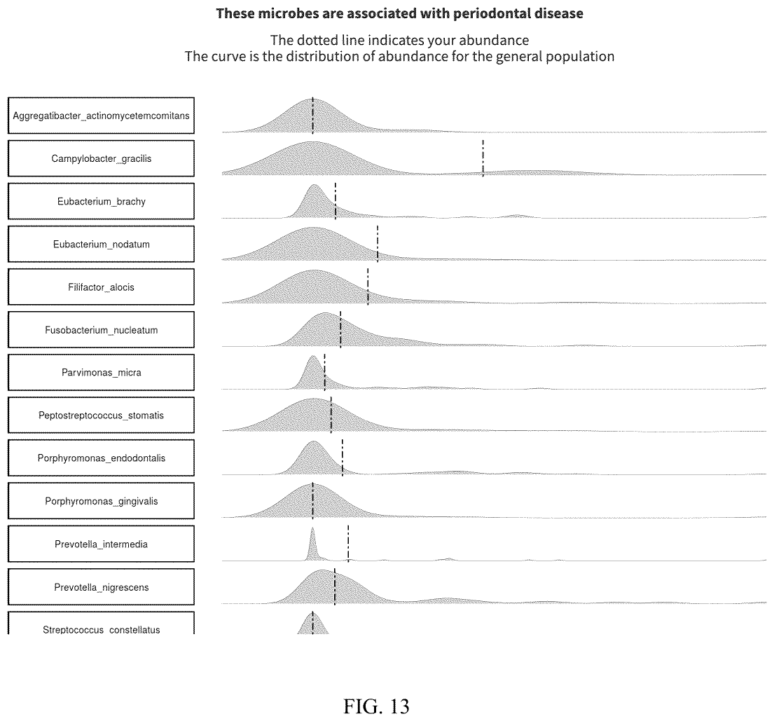

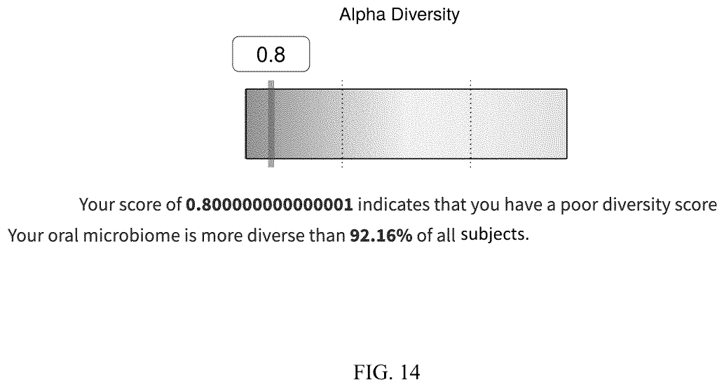

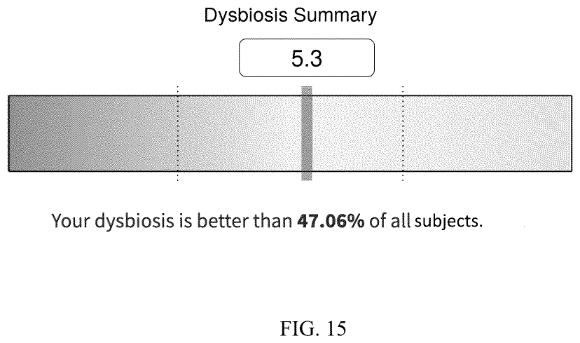

In various embodiments, the oral microbiome profile may be represented and visualized through a number of graphs and plots including, but not limited to, bar graphs (FIGS. 5 and 9), sunburst/pie charts (FIG. 6), tree diagrams (FIG. 7), scatter plots (FIG. 8), distribution curves (FIGS. 10-12), sliding scale plot (FIGS. 13 and 14), and tables (FIG. 15). In some instances, the oral microbiome profile of a subject may include the microbiome composition of the subject's oral cavity as determined by the abundancy values or relative abundances of the microbiomes in the oral cavity. In various embodiments, relative abundance is the count of microbial reads that come from each microbiome, and/or taxonomic group thereof, normalized to the total reads, so relative abundance may be represented as a percentage, fraction, or other data point. FIG. 5 shows a bar graph of a subject's oral microbiome profile including the relative abundance of microbiome species in the subject's oral cavity with respect to a healthy oral microbiome profile, and oral microbiome profiles associated with severe gum disease and dental caries. The lines represent individual species and the thickness denotes the relative abundance of those given species. As another example, FIG. 6 shows a sunburst chart of a subject's oral microbiome profile including the relative abundance of species of microbiomes in the subject's oral cavity. Sunburst charts may display a subject's oral microbiome profile with associated microbiome taxonomic group (e.g., species) ID and relative abundance data. Going from the middle out, FIG. 6 represents taxonomic profiling where the second ring and the outermost ring represent genus and species, respectively, of the microbiome profile, and the innermost ring represents the “oral microbiome” at 100% abundance. The example embodiment shown in FIG. 6 illustrates a slice of the middle ring representing about 6.8% relative abundance of organisms (e.g., genus) related to periodontal disease. Further, the slices in the outermost ring that fall within that slice in the middle ring represent about 0.4% relative abundance of Treponema denticola in the subject's oral cavity. FIG. 7 shows another example illustration of microbiome profile visualization using a tree diagram, where each branch of the tree diagram represents the abundance of different strains, species, genus, family, etc., of the microbiome of a subject.

In various embodiments, the visualization of a subject's microbiome profile may compare the oral microbiome profile of that subject to those of other subjects, allowing a patient to be better informed about the status of their oral health. For example, FIG. 8 shows a table comparing a subject's microbiome distribution to those of a group of subjects or a population. The table lists example abundance values (e.g., in percent) of microbiome species associated with good oral hygiene compared to the average abundances of those same microbiome species in the group of subjects.

With reference to FIG. 9, the relative dysbiosis score of a subject may be shown with respect to those of other subjects, allowing the subject to understand the status of their oral health compared to a large group of subjects. Dysbiosis is the measure of microbial imbalance between healthy and harmful microbes, and the dysbiosis score can be calculated as follows:

$$dysbiosis score = \frac{abundance of potentially pathogenic microbes}{abundance of normal microbes}$$

A higher or lower dysbiosis score may indicate that the subject in general has healthy or unhealthy, respectively, oral cavity. In some instances, however, the subject may benefit from understanding her/his relative oral health, i.e., the oral health of the subject in comparison to a suitable group of subjects (e.g., the general population). FIG. 9 shows an example plot of the relative dysbiosis score of a subject compared to the dysbiosis scores of the group of subjects. The plot also includes indications of oral diseases, if any, from which the subjects are suffering, allowing the subject to understand if any oral disease suffered by the subject is common to the group of subjects. The plot also allows the subject to be informed of oral diseases that may be attendant to the dysbiosis scores of the group of subjects.

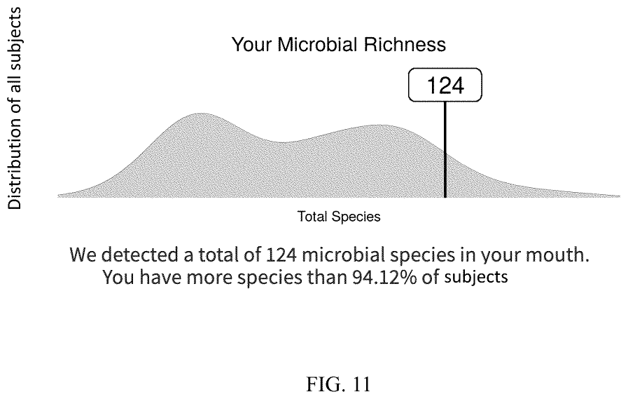

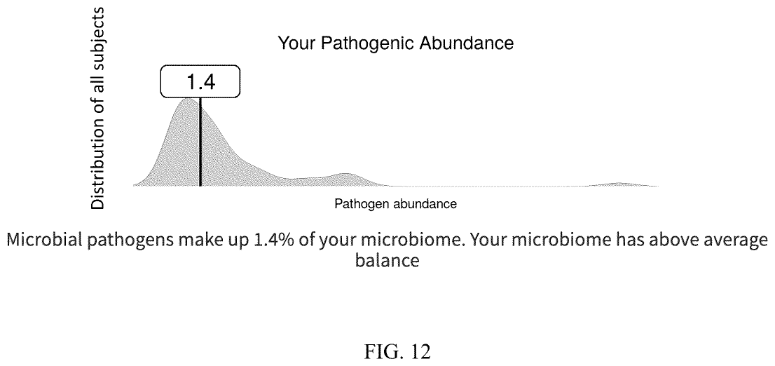

FIGS. 10-15 show additional non-limiting examples of representations of the oral microbiome profile of a subject with respect to those of a large group of subjects. FIG. 10 shows comparisons, between the subject and a group of other subjects (e.g., ten subjects), of microbial species associated with healthy oral cavities and oral cavities with periodontal disease. FIG. 11 identifies the microbial species richness, i.e., the total abundance of microbiome species, in the oral microbiome profile of the subject in comparison to other subjects. It is to be understood that representations of the oral microbiome profile as shown in FIG. 11 are not limited to microbial species, and that microbial richness of families, genus, strains, etc., of the subject's microbiome can also be plotted with respect to a large group of subjects. FIG. 12 identifies the abundance of pathogenic microbes of the subject with respect to the large group of subjects. FIG. 13 provides a breakdown of the total pathogenic microbes that are associated with an oral disease (e.g., periodontal disease) and identifies, for each pathogenic microbe, the abundance of that pathogenic microbe of the subject with respect to the large group of subjects.

FIG. 14 depicts a sliding scale plot indicating the microbiome diversity score of the subject in comparison to those of the group of subjects. In various embodiments, diversity scores can be derived from the number of unique microbial species and their cumulative abundance in proportion to the oral microbiome in order to convey the level of microbiome variance within the oral cavity of the subject and as compared to the aggregate (e.g., the group of subjects). That is, a subject's diversity score shows the number of unique microbes in a user's oral microbiome, and can be compared to the average diversity found across all users to characterize the subject's oral microbiome diversity with respect to a group of subjects. Diversity scores may be calculated using the Shannon index, taking into account two factors: the total number of species and the relative abundance of each species.

FIG. 15 depicts a sliding scale plot indicating the microbiome dysbiosis score of the subject in comparison to those of the group of subjects. As mentioned above, in various embodiments, dysbiosis is a measure of microbial imbalance between healthy and harmful microbes, and the degree of dysbiosis can be characterized by a dysbiosis score that incorporates the relative abundance of harmful or pathogenic microbes (e.g., and in some instances, microbe virulence factors, pathway analysis-based pathogenic microbe determinations, and/or the like). In some instances, the microbe taxonomic groups (e.g., species) that are considered in the computation of the dysbiosis score may be those that have a relative abundance exceeding a relative threshold abundance level. For example, the relative threshold abundance of a microbe species may be in the range from about 0.005% to about 1.5%, from about 0.0075% to about 1.25%, from about 0.01% to about 1%, from about 0.05% to about 0.5%, about 0.1%, including values and subranges therebetween.

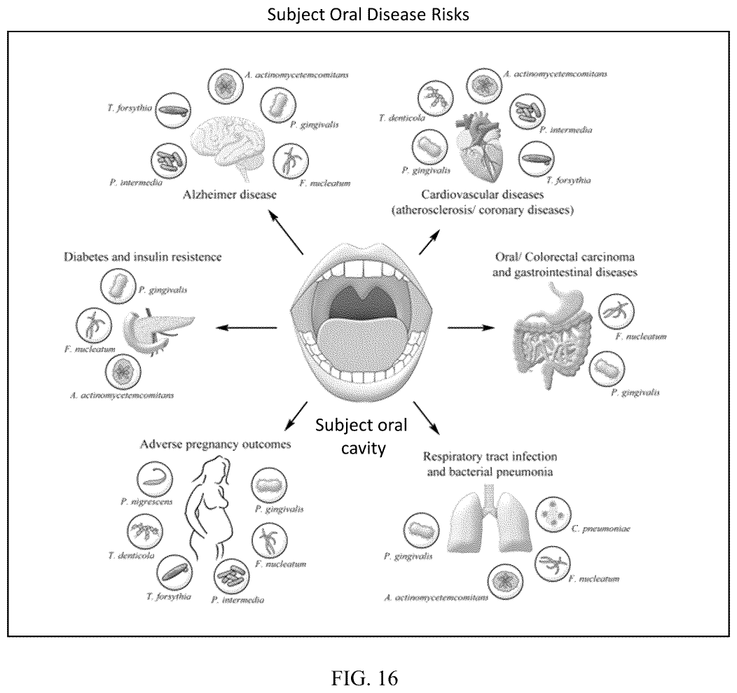

In various embodiments, the afore-mentioned subject report comprising the oral microbiome profile of a subject may also include information directed to the correlation between the oral microbiome profile of the subject and various diseases, providing the subject an understanding of the overall health risks that the subject may face. FIG. 16 shows a visualization of the correlations or associations between the subject's oral health (e.g., as characterized by the subject's oral microbiome profile) and the clinical risk factors faced by the subject as determined by their correlation to the subject's oral microbiome profile (e.g., Alzheimer's diseases, cardiovascular diseases (e.g., atherosclerosis), coronary disease, etc.), cancers (e.g., oral carcinoma, colorectal carcinoma), respiratory tract infections, pneumonia, diabetes, etc. In some instances, the subject report may also identify relationship between oral health and behavioral risk factors that may influence oral or overall health such as pregnancy status, diet of the subject, hygiene regimen, etc.

### III. Experimental Demonstration of Disclosed Methods

A study was conducted to experimentally demonstrate the applicability of the disclosed methods and systems for determining the oral microbiome profile or composition of subjects. In particular, the study was designed to assess the accuracy of the disclosed methods and systems to classify dental caries and periodontal disease based solely on the composition of the salivary oral microbiome. One hundred adult patients (18 years or older) were recruited to participate in a research study, where they were evaluated for cavities by radiography, visual inspection, or tactile probing, and for periodontal disease by pocket depth. Periodontal disease was defined as pocket depth >4 mm at any single position. Unstimulated patient saliva was collected prior to any treatment or dental cleaning.

To profile the oral microbiomes of the saliva samples from the patients, total genomic DNA was extracted from the saliva samples using a Zymobiomics DNA isolation kit. DNA was quality controlled to ensure high-quality DNA isolation. Purified DNA was subjected to in-house library preparation methods to generate Illumina sequencing libraries, which were quality controlled by fragment analysis and pooled equimolar for sequencing. Pooled libraries were sequenced on a NovaSeq to at least 5 million paired-end reads per sample at 150 bp length.

The oral microbial sequencing data was received in FASTQS file format, which were downsampled by seqtk to 5M reads and aligned to a human genome reference sequence (hg38) to remove human reads. The remaining reads were processed to identify and quantify the relative abundance of bacterial species. The processing included mapping the raw sequencing data to over 30,000 bacterial reference genomes to measure the relative abundance of bacterial strains, including those classified as periodontal and dental caries pathogens. An algorithm which takes into consideration the relative abundance of each bacterial strain, was used to compute a risk score for both caries and gum disease. The algorithm weighs the abundance of disease- and healthy-associated bacterial strains backed by internal studies and peer-reviewed research articles.

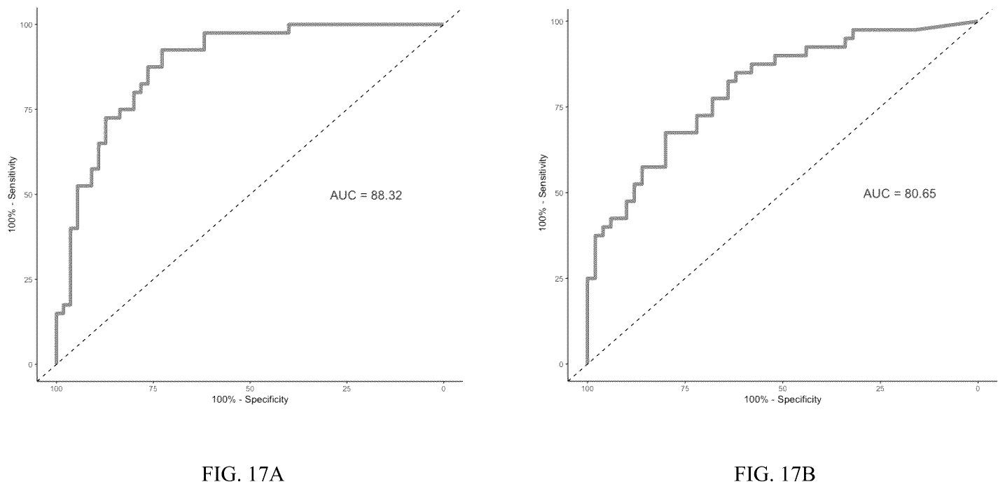

The accuracy of the disclosed methods and systems in classifying dental caries and periodontal disease was assessed using a receiver-operating curve (ROC), where the area under the curve (AUC) is used as the measure of the accuracy. An AUC of 1.0 indicates perfect accuracy at discriminating between classes (i.e., dental caries and periodontal disease), while an AUC of 0.5 is essentially random. FIGS. 17A-17B show for the ROCs for periodontal disease (FIG. 17A) and dental caries (FIG. 17B), where with respect to the former AUC=0.88, with 74% sensitivity and 85% specificity, and with respect to the latter AUC=0.81, with 69% sensitivity and 75% specificity. FIGS. 17A-17B demonstrate that the disclosed methods and system are capable of achieving excellent microbiome profiling of oral salivary samples, having exhibited best-in-class sensitivity and specificity for an oral health salivary microbiome test.

### IV. Computer-Implemented Systems

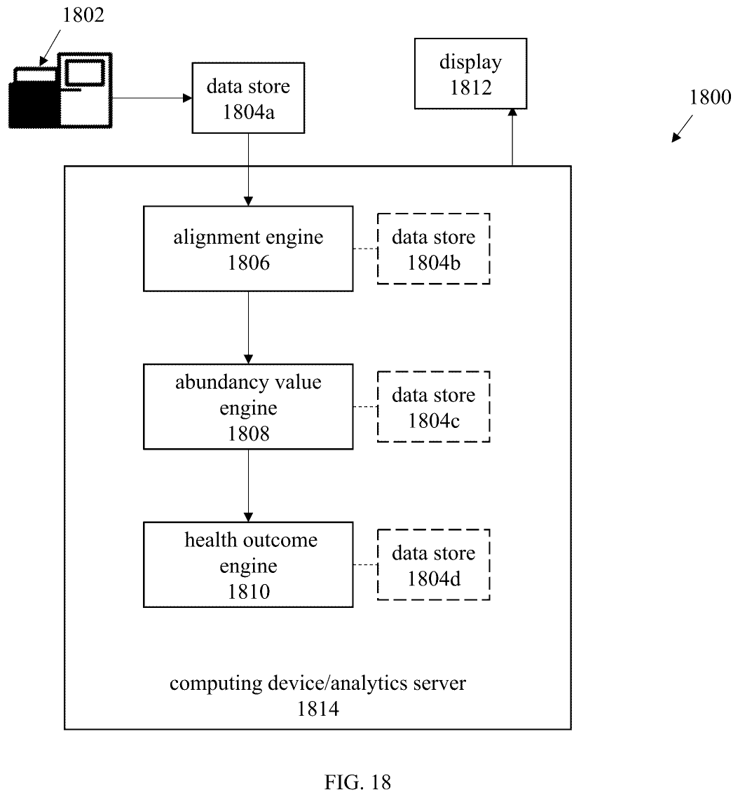

FIG. 18 is a schematic diagram 1800 of a computer device/analytics server 1814 for processing sequencing data derived from an oral sample according to various embodiments. In various embodiments, as discussed above, the oral sample of a subject may be processed to extract or generate the oral genomic/microbial sequencing data. In some aspects, the processing may be performed by a sequencing platform or engine 1802, which then stores the oral microbial sequencing data in data store 1804a. In various embodiments, the computer device/analytics server 1814 may include an alignment engine 1806 that is configured to receive the oral microbial sequencing data from data store 1804a, and align the oral microbial sequencing data received from data store 1804a to a reference genome in the data store 1804b to gather microbial reads from the microbial sequencing data. In some instances, the alignment engine 1806 may be configured to perform step 404 of the process of FIG. 4.

In various embodiments, the gathered microbial reads may be mapped to a database of microbiome type-specific (e.g., species specific) marker-genes, and an abundancy value engine 1808 of the computer device/analytics server 1814 may compute or generate an abundancy value for genus, specie, strain, family, etc., of a microbiome (e.g., bacteria, fungus, virus, etc., combinations thereof). In some instances, the generated abundancy values may be stored as output files in data store 1804c. For example, the output files can include relative abundance of a microbiome, which is the count of microbial reads that come from that microbe normalized to the total reads. Further, the abundancy value engine 1808 compares the computed abundancy values for each microbiome type to respective threshold values that are also stored in data store 1804c. In some instances, the abundancy value engine 1808 may be configured to perform steps 408 and 410 of the process of FIG. 4.

In various embodiments, the computer device/analytics server 1814 may include a health outcome engine 1810 that is configured to determine a health outcome for the subject of the oral sample based on the comparisons of the abundancy values to the respective threshold values. Further, the health outcome engine 1810 may compile an intervention plan, which can be behavioral interventions (e.g., related to lifestyle (e.g., maintaining proper diet), hygiene (e.g., proper use of oral care products (e.g., toothpaste, floss, mouthwash, probiotics, etc.)), and/or clinical (e.g., therapeutics). Such intervention plans as well as the afore-mentioned data related to the oral microbiome of the subject may be compiled in a subject report that is stored in data store 1804d. In some instances, the health outcome engine 1810 may be configured to perform step 412 of the process of FIG. 4.

In various embodiments, the computer device/analytics server 1814 may be coupled to a display 1812 that is configured to visualize or display the microbiome profiles in formats such as but not limited to tables, charts, graphs, and/or the like.

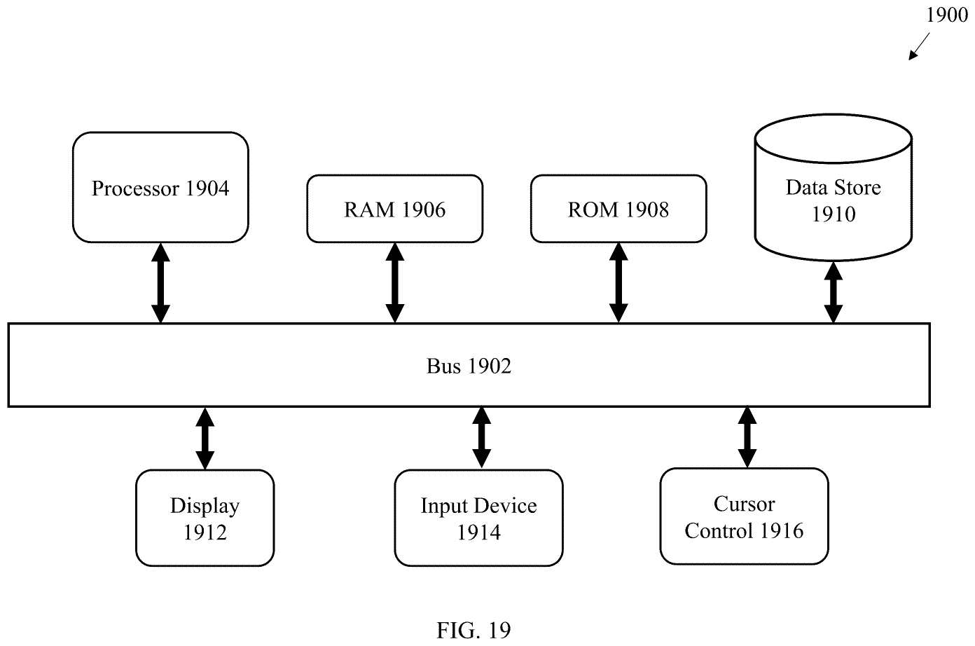

FIG. 19 is a block diagram of a computer system in accordance with various embodiments. Computer system 1900 may be an example of one implementation for the sequencing platform or engine 1802, the alignment engine 1806, the abundancy value engine 1808, the health outcome engine 1810, display 1812, and/or the like. In one or more examples, computer system 1900 can include a bus 1902 or other communication mechanism for communicating information, and a processor 1904 coupled with bus 1902 for processing information. In various embodiments, computer system 1900 can also include a memory, which can be a random-access memory (RAM) 1906 or other dynamic storage device, coupled to bus 1902 for determining instructions to be executed by processor 1904. Memory also can be used for storing temporary variables or other intermediate information during execution of instructions to be executed by processor 1904. In various embodiments, computer system 1900 can further include a read only memory (ROM) 1908 or other static storage device coupled to bus 1902 for storing static information and instructions for processor 1904. A storage device 1910, such as a magnetic disk or optical disk, can be provided and coupled to bus 1902 for storing information and instructions.

In various embodiments, computer system 1900 can be coupled via bus 1902 to a display 1912, such as a cathode ray tube (CRT) or liquid crystal display (LCD), for displaying information to a computer user. An input device 1914, including alphanumeric and other keys, can be coupled to bus 1902 for communicating information and command selections to processor 1904. Another type of user input device is a cursor control 1916, such as a mouse, a joystick, a trackball, a gesture input device, a gaze-based input device, or cursor direction keys for communicating direction information and command selections to processor 1904 and for controlling cursor movement on display 1912. This input device 1914 typically has two degrees of freedom in two axes, a first axis (e.g., x) and a second axis (e.g., y), that allows the device to specify positions in a plane. However, it should be understood that input devices 1914 allowing for three-dimensional (e.g., x, y and z) cursor movement are also contemplated herein.

Consistent with certain implementations of the present teachings, results can be provided by computer system 1900 in response to processor 1904 executing one or more sequences of one or more instructions contained in RAM 1906. Such instructions can be read into RAM 1906 from another computer-readable medium or computer-readable storage medium, such as storage device 1910. Execution of the sequences of instructions contained in RAM 1906 can cause processor 1904 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1904 for execution. Such a medium can take many forms, including but not limited to, non-volatile media, volatile media, and transmission media. Examples of non-volatile media can include, but are not limited to, optical, solid state, magnetic disks, such as storage device 1910. Examples of volatile media can include, but are not limited to, dynamic memory, such as RAM 1906. Examples of transmission media can include, but are not limited to, coaxial cables, copper wire, and fiber optics, including the wires that comprise bus 1902.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1904 of computer system 1900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flowcharts, diagrams, and accompanying disclosure can be implemented using computer system 1900 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1900, whereby processor 1904 would execute the analyses and determinations provided by these engines, subject to instructions provided by any one of, or a combination of, the memory components RAM 1906, ROM, 1908, or storage device 1910 and user input provided via input device 1914.

### Recitations of Various Embodiments of the Present Disclosure

Embodiment 1: A method for determining the oral microbiome composition of a subject and determining health outcomes, the method comprising: receiving, by one or more processors, oral microbial sequencing data of one or more subjects; aligning, by the one or more processors, the oral microbial sequencing data to a reference genome to count microbial reads from the oral microbial sequencing data; mapping, by the one or more processors, the microbial reads to a database of microbiome biomarkers; generating, by the one or more processors, an abundancy value for one or more of the microbiome biomarkers; comparing, by the one or more processors, the abundancy value for the one or more of the microbiome biomarkers to a threshold value for the one or more of the microbiome biomarkers; and determining a health outcome based on the comparison.

Embodiment 2: The method of embodiment 1, further comprising: processing an oral sample from the one or more subjects to generate the oral microbial sequencing data, the processing including: collecting an oral sample from the one or more subjects; purifying the oral sample to concentrate host and microbial nucleotides; depleting a portion of the host nucleotides; creating a sequencing library using the microbial nucleotides; and generating the oral microbial sequencing data from the sequencing library.

Embodiment 3: The method of embodiment 2, wherein the depleting the portion of the host nucleotides includes size selection.

Embodiment 4: The method of embodiment 2 or 3, wherein the creating the sequencing library using the microbial nucleotides includes fragmentating the microbial nucleotides and attaching unique barcodes thereto.

Embodiment 5: The method of any one of the preceding embodiments, further comprising: demultiplexing the oral microbial sequencing data to identify a source subject using the one or more processors.

Embodiment 6: The method of any one of the preceding embodiments, further comprising: identifying, using the one or more processors, a single microbial strain.

Embodiment 7: The method of any one of the preceding embodiments, further comprising: identifying a relative abundance between a plurality of microbes using the one or more processors.

Embodiment 8: The method of any one of the preceding embodiments, further comprising: generating a diversity score for a microbiome based on the abundancy value for the one or more of the microbiome biomarkers using the one or more processors.

Embodiment 9: The method of any one of the preceding embodiments, further comprising: generating a dysbiosis score with the abundancy value for the one or more of the microbiome biomarkers using the one or more processors.

Embodiment 10: The method of embodiment 9, further comprising: presenting the diversity score and/or the dysbiosis score in the form of interactive charts, plots, and/or graphs.

Embodiment 11: The method of embodiment 10, wherein the presenting includes displaying a profile for each of the one or more subjects, including the diversity score and the dysbiosis score.

Embodiment 12: The method of embodiment 9, wherein the generating the dysbiosis score and/or the diversity score includes: comparing, using the one or more processors, the abundancy value from one of the one or more subjects to previously generated abundancy values and clinical data from other subjects stored in a memory.

Embodiment 13: The method of embodiment 9, wherein the generating the dysbiosis score and/or the diversity score includes: comparing, using the one or more processors, the abundancy value from one of the one or more subjects to published journal and clinical data.

Embodiment 14: The method of embodiment 11, wherein the presenting includes displaying health guidance comprising lifestyle changes, hygiene changes, and/or therapeutics based on the profile.

Embodiment 15: The method of any one of the preceding embodiments, wherein the oral sample includes a salivary sample.

Embodiment 16: The method of any one of the preceding embodiments, further comprising: indicating a negative health outcome when the abundancy value exceeds the threshold value.

Embodiment 17: The method of embodiment 16, wherein the negative health outcome includes dental caries, halitosis, and/or periodontitis.

Embodiment 18: The method of any of embodiments 1-17, wherein the abundancy value is for commensal bacteria biomarker, the method further comprising: indicating a negative health outcome when the abundancy value is lower than the threshold value.

Embodiment 19: The method of any one of the preceding embodiments, wherein the microbiome biomarkers include biomarkers specific to a taxonomic group of the microbiome.

Embodiment 20: The method of embodiment 19, wherein the taxonomic group of the microbiome includes a specie of the microbiome.

Embodiment 21. A non-transitory computer-readable medium (CRM) having stored thereon computer-readable instructions executable to cause performance of any of the methods of embodiments 1-20.

Embodiment 22. A system for determining an oral microbiome composition within an oral cavity to determine potential health outcomes, the system comprising: a data store configured to store oral microbial sequencing data from one or more subjects, a reference genome, and a database of microbiome biomarkers; and a computer device communicatively connected to the data store and including an alignment engine, an abundancy value engine, and a health outcome engine, the computer device configured to read the instructions from the data store to cause the system to perform any of the methods of embodiments 1-20.

1. A method for determining an oral microbiome composition of a subject and determining health outcomes, the method comprising:
receiving, by one or more processors, oral microbial sequencing data of one or more subjects;
aligning, by the one or more processors, the oral microbial sequencing data to a reference genome to count microbial reads from the oral microbial sequencing data;
mapping, by the one or more processors, the microbial reads to a database of microbiome biomarkers;
generating, by the one or more processors, an abundancy value for one or more of the microbiome biomarkers based on the mapping;
comparing, by the one or more processors, the abundancy value for the one or more of the microbiome biomarkers to a threshold value for the one or more of the microbiome biomarkers; and
determining a health outcome based on the comparison.

2. The method of claim 1, further comprising:
processing an oral sample from the one or more subjects to generate the oral microbial sequencing data, the processing including:
collecting the oral sample from the one or more subjects;
purifying the oral sample to concentrate host and microbial nucleotides;
depleting a portion of the host nucleotides;
creating a sequencing library using the microbial nucleotides; and
generating the oral microbial sequencing data from the sequencing library.

3. The method of claim 2, wherein the depleting the portion of the host nucleotides includes size selection.

4. The method of claim 2, wherein the creating the sequencing library using the microbial nucleotides includes fragmentating the microbial nucleotides and attaching unique barcodes thereto.

5. The method of claim 1, further comprising:
demultiplexing the oral microbial sequencing data to identify a source subject using the one or more processors.

6. The method of claim 1, further comprising:
identifying, using the one or more processors, a single microbial strain.

7. The method of claim 1, further comprising:
identifying a relative abundance between a plurality of microbes using the one or more processors.

8. The method of claim 1, further comprising:
generating a diversity score for a microbiome based on the abundancy value for the one or more of the microbiome biomarkers using the one or more processors.

9. The method of claim 1, further comprising:
generating a dysbiosis score with the abundancy value for the one or more of the microbiome biomarkers using the one or more processors.

10. The method of claim 9, further comprising:
presenting the diversity score and/or the dysbiosis score in the form of interactive charts, plots, and/or graphs.

11. The method of claim 10, wherein the presenting includes displaying a profile for each of the one or more subjects, including the diversity score and the dysbiosis score.

12. The method of claim 9, wherein the generating the dysbiosis score and/or the diversity score includes:
comparing, using the one or more processors, the abundancy value from one of the one or more subjects to previously generated abundancy values and clinical data from other subjects stored in a memory.

13. The method of claim 9, wherein the generating the dysbiosis score and/or the diversity score includes:
comparing, using the one or more processors, the abundancy value from one of the one or more subjects to published journal and clinical data.

14. The method of claim 11, wherein the presenting includes displaying health guidance comprising lifestyle changes, hygiene changes, and/or therapeutics based on the profile.

15. The method of claim 1, wherein the oral sample includes a salivary sample.

16. The method of claim 1, further comprising:
indicating a negative health outcome when the abundancy value exceeds the threshold value.

17. The method of claim 16, wherein the negative health outcome includes dental caries, halitosis, and/or periodontitis.

18. The method of claim 1, wherein the abundancy value is for commensal bacteria biomarker, the method further comprising:
indicating a negative health outcome when the abundancy value is lower than the threshold value.

19. The method of claim 1, wherein the microbiome biomarkers include microbiome biomarkers specific to a taxonomic group of the microbiome.

20. The method of claim 19, wherein the taxonomic group of the microbiome includes a specie of the microbiome.

21. A non-transitory computer-readable medium (CRM) in which a program is stored for causing a computer to perform a method for determining the oral microbiome composition, the method comprising:
receiving, by one or more processors, oral microbial sequencing data of one or more subjects;
aligning, by the one or more processors, the oral microbial sequencing data to a reference genome to count microbial reads from the oral microbial sequencing data;
mapping, by the one or more processors, the microbial reads to a database of microbiome biomarkers;
generating, by the one or more processors, an abundancy value for one or more of the microbiome biomarkers based on the mapping;
comparing, by the one or more processors, the abundancy value for the one or more of the microbiome biomarkers to a threshold value for the one or more of the microbiome biomarkers; and
determining a health outcome based on the comparison.

22. A system for determining an oral microbiome composition within an oral cavity to determine potential health outcomes, the system comprising:
a data store configured to store oral microbial sequencing data from one or more subjects, a reference genome, and a database of microbiome biomarkers; and
a computer device communicatively connected to the data store, the computer device comprising:
an alignment engine configured to:
receive the oral microbial sequencing data of the one or more subjects;
align the oral microbial sequencing data to the reference genome to count microbial reads from the oral microbial sequencing data; and
map the microbial reads to the database of microbiome biomarkers;
an abundancy value engine configured to:
generate an abundancy value for one or more of the microbiome biomarkers based on the mapping; and
a health outcome engine configured to:
compare the abundancy value for the one or more of the microbiome biomarkers to a threshold value for the one or more of the microbiome biomarkers; and
determine a health outcome based on the comparison.
